(19) United States
(12) Patent Application Publication
(10) Pub. No.: US 2010/0003203 A1
(43) Pub. Date: Jan. 7, 2010

(54) METHOD OF PRODUCING SURFACE-MODIFIED NANOPARTICULATE METAL OXIDES, METAL HYDROXIDES AND/OR METAL OXYHYDROXIDES

(75) Inventors: Andrey Karpov, Mannheim (DE); Hartmut Hibst, Schriesheim (DE); Jutta Kissel, Herxheim (DE); Bernd Bechtloff, Ludwigshafen (DE); Hartwig Voss, Frankenthal (DE); Kerstin Schierle-Arndt, Zwingenberg (DE); Valerie Andre, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/445,166

(22) PCT Filed: Oct. 10, 2007
(86) PCT No.: PCT/EP07/60778

§ 371 Date: Apr. 10, 2009

(30) Foreign Application Priority Data
Oct. 11, 2006 (EP) 06122082.8

(51) Int. Cl. A61K 8/19; A01N 59/00; A01N 59/20; A01N 59/16; C08K 3/22; A61Q 17/04; C01F 7/02; C01F 5/02; C01F 5/14; C01F 17/00; C01G 49/02; C01G 45/02; C01G 53/04; C01G 51/04; C01G 3/02; C01G 25/02; C01G 23/04; C01G 9/02

(52) U.S. Cl. 424/59; 424/600; 424/630; 424/639; 424/641; 424/646; 424/682; 524/413; 524/430; 524/431; 524/432; 524/433; 423/625; 423/629; 423/636; 423/263; 423/632; 423/605; 423/594.19; 423/604; 423/608; 423/622

(57) ABSTRACT

The present invention relates to methods of producing surface-modified nanoparticulate particles at least of one metal oxide, metal hydroxide and/or metal oxide hydroxide, and aqueous suspensions of these particles. The invention further relates to the surface-modified nanoparticulate particles, obtainable by these methods, at least of one metal oxide, metal hydroxide and/or metal oxide hydroxide and aqueous suspensions of these particles, and to their use for cosmetic sunscreen preparations, as stabilizer in plastics and as antimicrobial active ingredient.

The present invention relates to methods of producing surface-modified nanoparticulate particles at least of one metal oxide, metal hydroxide and/or metal oxide hydroxide, and aqueous suspensions of these particles. The invention further relates to the surface-modified nanoparticulate particles, obtainable by these methods, at least of one metal oxide, metal hydroxide and/or metal oxide hydroxide and aqueous suspensions of these particles, and to their use for cosmetic sunscreen preparations, as stabilizer in plastics and as antimicrobial active ingredient.

Metal oxides are used for diverse purposes, thus, for example, as white pigment, as catalyst, as constituent of antibacterial skin protection salves and as activator for the vulcanization of rubber. Finely divided zinc oxide or titanium dioxide as UV-absorbing pigments are found in cosmetic sunscreen compositions.

Nanoparticles is the term used to refer to particles in the nanometers order of magnitude. Being the size they are, they lie in the transition range between atomic or monomolecular systems and continuous macroscopic structures. Besides their mostly very large surface, nanoparticles are characterized by particular physical and chemical properties which differ significantly from those of larger particles. Thus, nanoparticles often have a lower melting point, absorb light only at relatively short wavelengths and have different mechanical, electrical and magnetic properties to macroscopic particles of the same material. By using nanoparticles as building blocks, it is possible to use many of these special properties also for macroscopic materials (Winnacker/Küchler, Chemische Technik Prozesse und Produkte, (ed.: R. Dittmayer, W. Keim, G. Kreysa, A. Oberholz), Vol. 2: Neue Technologien, Chapter 9, Wiley-VCH Verlag 2004).

Within the scope of the present invention, the term “nanoparticles” refers to particles with an average diameter of from 1 to 500 nm, determined by means of electron microscopic methods.

Nanoparticulate zinc oxide with particle sizes below about 100 nm is potentially suitable for use as UV absorber in transparent organic-inorganic hybrid materials, plastics, paints and coatings. In addition, a use to protect UV-sensitive organic pigments is also possible.

Particles, particle aggregates or agglomerates of zinc oxide which are larger than about 100 nm lead to scattered-light effects and thus to undesired decrease in transparency in the visible light region. For this reason, the redispersibility, i.e. the ability to convert the produced nanoparticulate zinc oxide into a colloidally disperse state, is an important prerequisite for the abovementioned applications.

Nanoparticulate zinc oxide with particle sizes below about 5 nm exhibit, on account of the size quantization effect, a blue shift in the absorption edge (L. Brus, J. Phys. Chem. (1986), 90, 2555-2560) and are therefore less suitable for use as UV absorbers in the UV-A region.

The production of finely divided metal oxides, for example zinc oxide, by dry and wet processes is known. The classical method of burning zinc, which is known as the dry process (e.g. Gmelin Volume 32, 8th Edition, supplementary volume, p. 772ff), produces aggregated particles having a broad size distribution. Although in principle it is possible to produce particle sizes in the submicrometer range by grinding procedures, because the shear forces which can be achieved are too low, dispersions with average particle sizes in the lower nanometer range are obtainable from such powders only with very great expenditure. Particularly finely divided zinc oxide is produced primarily by wet chemical methods by precipitation processes. Precipitation in aqueous solution generally gives hydroxide- and/or carbonate-containing materials which have to be thermally converted to zinc oxide. The thermal aftertreatment here has an adverse effect on the finely divided nature since the particles are subjected during this treatment to sinter processes which lead to the formation of micrometer-sized aggregates which can be broken down only incompletely again to the primary particles by grinding.

Nanoparticulate metal oxides can, for example, be obtained by the microemulsion process. In this process, a solution of a metal alkoxide is added dropwise to a water-in-oil microemulsion. In the inverse micelles of the microemulsion, the size of which is in the nanometer range, then takes place the hydrolysis of the alkoxides to the nanoparticulate metal oxide. The disadvantages of this process are particularly that the metal alkoxides are expensive starting materials, that additionally emulsifiers have to be used and that the production of the emulsions with droplet sizes in the nanometer range is a complex process step.

DE 199 07 704 describes a nanoparticulate zinc oxide produced by a precipitation reaction. In the process, the nanoparticulate zinc oxide is produced starting from a zinc acetate solution via an alkaline precipitation. The centrifuged-off zinc oxide can be redispersed to a sol by adding methylene chloride. The zinc oxide dispersions produced in this way have the disadvantage that, because of the lack of surface modification, they do not have good long-term stability.

WO 00/50503 describes zinc oxide gels which comprise nanoparticulate zinc oxide with a particle diameter of ≦15 nm and which are redispersible to sols. Here, the solids produced by basic hydrolysis of a zinc compound in alcohol or in an alcohol/water mixture are redispersed by adding dichloromethane or chloroform. The disadvantage here is that stable dispersions are not obtained in water or in aqueous dispersants.

In the publication from Chem. Mater. 2000, 12, 2268-74 “Synthesis and Characterization of Poly(vinylpyrrolidone)-Modified Zinc Oxide Nanoparticles” by Lin Guo and Shihe Yang, zinc oxide nanoparticles are surface-coated with polyvinylpyrrolidone. The disadvantage here is that zinc oxide particles coated with polyvinylpyrrolidone are not dispersible in water.

WO 93/21127 describes a method of producing surface-modified nanoparticulate ceramic powders. Here, a nanoparticulate ceramic powder is surface-modified by applying a low molecular weight organic compound, for example propionic acid. This method cannot be used for the surface modification of zinc oxide since the modification reactions are carried out in aqueous solution and zinc oxide dissolves in aqueous organic acids. For this reason, this method cannot be used for producing zinc oxide dispersions; moreover, zinc oxide is not mentioned in this application either as a possible starting material for nanoparticulate ceramic powders.

WO 02/42201 describes a method of producing nanoparticulate metal oxides in which dissolved metal salts are thermally decomposed in the presence of surfactants. The decomposition takes place under conditions under which the surfactants form micelles; furthermore, depending on the metal salt chosen, temperatures of several hundred degrees Celsius may be required in order to achieve the decomposition. The method is therefore very costly in terms of apparatus and energy.

In a publication in Materials Letters 57 (2003), pp. 4079-4082, P. Si et al. describe the production of nanoparticulate zinc oxide rods through joint grinding of solid zinc acetate with sodium hydroxide in the presence of polyethylene glycol as nonionic dispersant. However, the method is too complex for industrial application and the components are not as homogeneously mixed together as when the starting point is a solution.

In the publication in Inorganic Chemistry 42(24), 2003, pp. 8105-9, Z. Li et al. disclose a method of producing nanoparticulate zinc oxide rods by hydrothermal treatment of [Zn(OH)₄]²⁻ complexes in an autoclave in the presence of polyethylene glycol. However, autoclave technology is very complex and the rod-shaped habit of the products makes them unsuitable for applications on the skin.

WO 2004/052327 describes surface-modified nanoparticulate zinc oxides in which the surface modification comprises a coating with an organic acid. DE-A 10 2004 020 766 discloses surface-modified nanoparticulate metal oxides which have been produced in the presence of polyaspartic acid. EP 1455737 describes surface-modified nanoparticulate zinc oxides in which the surface modification comprises a coating with an oligo- or polyethylene glycolic acid. On account of the acids used, these products are not suitable for cosmetic applications since they possibly have only poor skin compatibility.

The object of the present invention was therefore to provide surface-modified nanoparticulate particles at least of one metal oxide, metal hydroxide and/or metal oxide hydroxide, and aqueous suspensions thereof where, with regard to cosmetic applications, particularly in the field of UV protection, the substances used for the surface modification should have good skin compatibility and ideally have already been trialed and approved as constituents of cosmetic preparations. A further object of the invention was the development of methods of producing these surface-modified nanoparticulate particles, and their aqueous suspensions and their use.

This object is achieved by surface-modified nanoparticulate particles at least of one metal oxide, metal hydroxide and/or metal oxide hydroxide which are precipitated from a solution in the presence of a nonionic dispersant.

The invention thus provides a method of producing surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, comprising the steps
a) producing a solution of water and at least one metal salt of the abovementioned metals (solution 1) and a solution of water and at least one strong base (solution 2), where at least one of the two solutions 1 and 2 comprises at least one nonionic dispersant whose chemical structure comprises between 2 and 10 000 —CH₂CH₂O— groups,
b) mixing the solutions 1 and 2 produced in step a) at a temperature in the range from 0 to 120° C., during which the surface-modified nanoparticulate particles are formed and precipitate out of the solution to form an aqueous suspension,
c) separating off the surface-modified nanoparticulate particles from the aqueous suspension obtained in step b), and
d) drying the surface-modified nanoparticulate particles obtained in step c).

The metal oxide, metal hydroxide and metal oxide hydroxide here may either be the anhydrous compounds or the corresponding hydrates.

The metal salts in process step a) may be metal halides, acetates, sulfates or nitrates or hydrates of the aforementioned salts. Preferred metal salts are halides, for example zinc chloride or titanium tetrachloride, acetates, for example zinc acetate dihydrate, and nitrates, for example zinc nitrate. A particularly preferred metal salt is zinc chloride or zinc nitrate.

The concentration of the metal salts in solution 1 is generally in the range from 0.05 to 1 mol/l, preferably in the range from 0.1 to 0.5 mol/l, particularly preferably 0.2 to 0.4 mol/l.

The strong bases to be used according to the invention may in general be any substances which are able to produce a pH of from about 8 to about 13, preferably of from about 9 to about 12.5, in aqueous solution depending on their concentration. These may, for example, be metal oxides or hydroxides, and ammonia or amines. Preference is given to using alkali metal hydroxides, such as sodium or potassium hydroxide, alkaline earth metal hydroxides, such as calcium hydroxide or ammonia. Particular preference is given to using sodium hydroxide, potassium hydroxide and ammonia. In a preferred embodiment of the invention, ammonia can also be formed in situ during process steps a) and/or b) as a result of the thermal decomposition of urea.

The concentration of the strong base in solution 2 produced in process step a) is generally chosen so that a hydroxyl ion concentration in the range from 0.1 to 2 mol/l, preferably from 0.2 to 1 mol/l and particularly preferably from 0.4 to 0.8 mol/l is established in solution 2. Preferably, the hydroxyl ion concentration in solution 2 (c(OH—)) is chosen depending on the concentration and the valence of the metal ions in solution 1 (c(Mⁿ⁺)), so that it obeys the formula

n·c(Mⁿ⁺)=c(OH⁻)

where c is a concentration and Mⁿ⁺ is at least one metal ion with the valence n. For example, in the case of a solution 1 with a concentration of divalent metal ions of 0.2 mol/l, preference is given to using a solution 2 with a hydroxyl ion concentration of 0.4 mol/l.

According to the invention, the nonionic dispersants are surface-active substances whose chemical structure comprises between 2 and 10 000 —CH₂CH₂O— groups, preferably between 3 and 200 —CH₂CH₂O— groups. These groups are formed, for example, by adding a corresponding number of ethylene oxide molecules onto hydroxyl or carboxyl group-containing substrates and generally form one or more connected ethylene glycol chains whose chemical structure corresponds to the formula —(—CH₂CH₂O—)ₙ— where n is from about 2 to about 80.

In a preferred embodiment of the invention, the nonionic dispersant used is at least one substance from one of the following groups:

Addition products of from 2 to 80 mol of ethylene oxide and, if appropriate, 1 to 15 mol of propylene oxide onto
linear fatty alcohols having 8 to 22 carbon atoms (e.g. cetylstearyl alcohol),
fatty acids having 12 to 22 carbon atoms,
alkylphenols having 8 to 15 carbon atoms in the alkyl group,
glycerol monoesters and diesters, sorbitol monoesters and diesters and sorbitan monoesters and diesters of saturated and unsaturated fatty acids having 6 to 22 carbon atoms,
alkyl monoglycosides and oligoglycosides having 8 to 22 carbon atoms in the alkyl radical,
castor oil and/or hydrogenated castor oil,
partial esters based on linear, branched, unsaturated or saturated fatty acids having 12 to 22 carbon atoms,
ricinoleic acid,
12-hydroxystearic acid,
acetic acid,
lactic acid,
glycerol,
polyglycerol,
pentaerythritol,
dipentaerythritol,
sucrose,
sugar alcohols (e.g. sorbitol),
alkyl glucosides (e.g. methyl glucoside, butyl glucoside, lauryl glucoside),
polyglucosides (e.g. cellulose),
wool wax alcohols having 24 to 36 carbon atoms,
Guerbet alcohols having 6 to 22 carbon atoms,
   and polyalkylene glycols whose structure comprises between 2 and 80 ethylene glycol units.

In a particularly preferred embodiment of the invention, the nonionic dispersant used is at least one substance from one of the following groups:

Addition products of from 2 to 80 mol of ethylene oxide onto
linear fatty alcohols having 8 to 22 carbon atoms,
alkylphenols having 8 to 15 carbon atoms in the alkyl group, and
castor oil and/or hydrogenated castor oil.

Many of the nonionic dispersants to be used according to the invention are commercially available under the trade name Cremophor® (BASF Aktiengesellschaft).

The ethylene oxide addition products can, in technical grade, always also comprise a small fraction of the substrates containing free hydroxyl or carboxyl groups listed above by way of example. As a rule, this fraction is less than 20% by weight, preferably less than 5% by weight, based on the total amount of the dispersant.

The concentration of the nonionic dispersants in solutions 1 and/or 2 produced in process step a) is usually in the range from 0.1 to 20 g/l, preferably from 1 to 10 g/l, particularly preferably from 1.5 to 5 g/l.

A preferred embodiment of the method according to the invention is one in which the precipitation of the metal oxide, metal hydroxide and/or the metal oxide hydroxide takes place in the presence of a nonionic dispersant which is obtained by reacting hydrogenated castor oil or fatty alcohols with about 35 to about 50 equivalents of ethylene oxide. In a particularly preferred embodiment of the invention, Cremophor® CO 40 (BASF Aktiengesellschaft), an addition product of 40 equivalents of ethylene oxide onto hydrogenated castor oil or Cremophor® A 25 (BASF Aktiengesellschaft), an addition product of 25 equivalents of ethylene oxide onto cetylstearyl alcohol, is used as nonionic dispersant.

The mixing of the two solutions 1 and 2 (aqueous metal salt solution and aqueous base solution) in process step b) takes place at a temperature in the range from 0° C. to 120° C., preferably in the range from 10° C. to 100° C., particularly preferably in the range from 15° C. to 80° C.

Depending on the metal salts used, the mixing can be carried out at a pH in the range from 3 to 13. In the case of zinc oxide, the pH during mixing is in the range from 7 to 11.

According to the invention, the time for the mixing of the two solutions in process step b) is in the range from 1 second to 6 hours, preferably in the range from 1 minute to 2 hours. In general, the mixing time in the case of the discontinuous procedure is longer than in the case of the continuous procedure.

The mixing in process step b) can take place, for example, by combining an aqueous solution of a metal salt, for example of zinc chloride or zinc nitrate, with an aqueous solution of a mixture of a nonionic dispersant and an alkali metal hydroxide or ammonium hydroxide, in particular sodium hydroxide. Alternatively, it is also possible to combine an aqueous solution of a mixture of a nonionic dispersant and a metal salt, for example of zinc chloride or zinc nitrate, with an aqueous solution of an alkali metal hydroxide or ammonium hydroxide, in particular of sodium hydroxide. Furthermore, an aqueous solution of a mixture of a nonionic dispersant and a metal salt, for example of zinc chloride or zinc nitrate, can also be combined with an aqueous solution of a mixture of a nonionic dispersant and an alkali metal hydroxide or ammonium hydroxide, in particular sodium hydroxide.

In a preferred embodiment of the invention, the mixing in process step b) takes place through metered addition of an aqueous solution of a mixture of a nonionic dispersant and an alkali metal hydroxide or ammonium hydroxide, in particular sodium hydroxide, to an aqueous solution of a metal salt, for example of zinc chloride or zinc nitrate, or through metered addition of an aqueous solution of an alkali metal hydroxide or ammonium hydroxide, in particular sodium hydroxide, to an aqueous solution of a mixture of a nonionic dispersant and a metal salt, for example of zinc chloride or zinc nitrate.

During mixing and/or after mixing, the surface-modified nanoparticulate particles are formed and precipitate out of the solution to form an aqueous suspension. Preferably, the mixing takes place with simultaneous stirring of the mixture. After completely combining the two solutions 1 and 2, the stirring is preferably continued for a time between 30 minutes and 5 hours at a temperature in the range from 0° C. to 120° C.

A further preferred embodiment of the method according to the invention is one where at least one of process steps a) to d) is carried out continuously. In the case of a continuously operated procedure, process step b) is preferably carried out in a tubular reactor.

Preferably, the continuous method is carried out such that the mixing in process step b) takes place in a first reaction space at a temperature T1, in which an aqueous solution 1 at least of one metal salt and an aqueous solution 2 at least of one strong base are continuously introduced, where at least one of the two solutions 1 and 2 comprises at least one nonionic dispersant whose chemical structure comprises between 2 and 10 000 —CH₂CH₂O— groups, from which the formed suspension is continuously removed and transferred to a second reaction space for heating at a temperature T2, during which the surface-modified nanoparticulate particles are formed.

As a rule, the continuous process is carried out such that the temperature T2 is higher than the temperature T1.

The methods described at the outset are particularly suitable for producing surface-modified nanoparticulate particles of titanium dioxide and zinc oxide, in particular of zinc oxide. In this case, the precipitation of the surface-modified nanoparticulate particles of zinc oxide takes place from an aqueous solution of zinc acetate, zinc chloride or zinc nitrate at a pH in the range from 8 to 13 in the presence of a nonionic dispersant.

A further advantageous embodiment of the method according to the invention is one in which the surface-modified nanoparticulate particles of a metal oxide, metal hydroxide and/or metal oxide hydroxide, in particular of zinc oxide, have a BET surface area in the range from 25 to 500 m²/g, preferably 30 to 400 m²/g, particularly preferably 40 to 300 m²/g.

The invention is based on the finding that a surface modification of nanoparticulate metal oxides, metal hydroxides and/or metal oxide hydroxides with nonionic dispersants can achieve long-term stability of dispersions of the surface-modified nanoparticulate metal oxides, in particular in cosmetic preparations, without undesired changes in the pH during storage of these preparations.

The precipitated particles can be separated off from the aqueous suspension in process step c) in a manner known per se, for example by filtration or centrifugation. If required, the aqueous dispersion can be concentrated prior to isolating the precipitated particles by means of a membrane process such as nano-, ultra-, micro- or crossflow filtration and, if appropriate, can be at least partially freed from undesired water-soluble constituents, for example alkali metal salts, such as sodium chloride or sodium nitrate.

It has proven to be advantageous to carry out the separation of the surface-modified nanoparticulate particles from the aqueous suspension obtained in step b) at a temperature in the range from 10 to 50° C., preferably at room temperature. It is therefore advantageous to cool, if appropriate, the aqueous suspension obtained in step b) to such a temperature.

In process step d), the filter cake obtained can be dried in a manner known per se, for example in a drying cabinet at temperatures between 40 and 100° C., preferably between 50 and 80° C., under atmospheric pressure to a constant weight.

The present invention further provides surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, and the surface modification comprises a coating with at least one nonionic dispersant obtainable by the method described at the outset.

The present invention further provides surface-modified nanoparticulate particles at least of one metal oxide, metal hydroxide and/or metal oxide hydroxide, in particular of zinc oxide, where the surface modification comprises a coating with a nonionic dispersant, with a BET surface area in the range from 25 to 500 m²/g, preferably 30 to 400 m²/g, particularly preferably 40 to 300 m²/g.

According to a preferred embodiment of the invention, the surface-modified nanoparticulate particles at least of one metal oxide, metal hydroxide and/or metal oxide hydroxide are coated with a nonionic dispersant which is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

According to a further preferred embodiment of the present invention, the surface-modified nanoparticulate particles have a diameter of from 10 to 200 nm. This is particularly advantageous since good redispersibility is ensured within this size distribution.

According to a particularly preferred embodiment of the present invention, the surface-modified nanoparticulate particles have a diameter of from 20 to 100 nm. This size range is particularly advantageous since, for example following redispersion of such zinc oxide nanoparticles, the resulting suspensions are transparent and thus do not affect the coloring when added to cosmetic formulations. Moreover, this also gives rise to the possibility of use in transparent films.

The present invention further provides the use of surface-modified nanoparticulate particles at least of one metal oxide, metal hydroxide and/or metal oxide hydroxide, in particular titanium dioxide or zinc oxide, which are produced by the method according to the invention as UV protectants in cosmetic sunscreen preparations, as stabilizer in plastics and as antimicrobial active ingredient.

According to a preferred embodiment of the present invention, the surface-modified nanoparticulate particles at least of one metal oxide, metal hydroxide and/or metal oxide hydroxide, in particular titanium dioxide or zinc oxide, are redispersible in a liquid medium and form stable suspensions. This is particularly advantageous because, for example, the suspensions produced from the zinc oxide according to the invention do not have to be dispersed again prior to further processing, but can be processed directly.

According to a preferred embodiment of the present invention, the surface-modified nanoparticulate particles at least of one metal oxide, metal hydroxide and/or metal oxide hydroxide are redispersible in polar organic solvents and form stable suspensions. This is particularly advantageous since, as a result of this, uniform incorporation, for example into plastics or films, is possible.

According to a further preferred embodiment of the present invention, the surface-modified nanoparticulate particles at least of one metal oxide, metal hydroxide and/or metal oxide hydroxide are redispersible in water, where they form stable suspensions. This is particularly advantageous since this opens up the possibility of using the material according to the invention for example in cosmetic formulations, where dispensing with organic solvents is a great advantage. Mixtures of water and polar organic solvents are also conceivable.

Since numerous applications of the surface-modified nanoparticulate particles according to the invention at least of one metal oxide, metal hydroxide and/or metal oxide hydroxide require them to be used in the form of an aqueous suspension, it is possible, if appropriate, to dispense with their isolation as solid.

The present invention therefore further provides a method of producing an aqueous suspension of surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, comprising the steps
a) producing a solution of water and at least one metal salt of the abovementioned metals (solution 1) and a solution of water and at least one strong base (solution 2), where at least one of the two solutions 1 and 2 comprises at least one nonionic dispersant whose chemical structure comprises between 2 and 10 000 —CH₂CH₂O— groups,
b) mixing the solutions 1 and 2 produced in step a) at a temperature in the range from 0 to 120° C., during which the surface-modified nanoparticulate particles are formed and precipitate out of the solution to form an aqueous suspension, and
c) if appropriate concentrating the formed aqueous suspension and/or separating off by-products.

For a more detailed description of the procedure for process steps a) and b), and the feed substances and process parameters used, reference is made to the statements made above.

If required, the aqueous suspension formed in step b) can be concentrated in process step c), for example if a higher solid content is desired. Concentration can be carried out in a manner known per se, for example by distilling off the water (at atmospheric pressure or at reduced pressure), filtration or centrifugation.

In addition, it may be required to separate off by-products from the aqueous suspension formed in step b) in process step c), namely when these would interfere with the further use of the suspension. By-products coming into consideration are primarily salts dissolved in water which are formed during the reaction according to the invention between the metal salt and the strong base besides the desired metal oxide, metal hydroxide and/or metal oxide hydroxide particles, for example sodium chloride, sodium nitrate or ammonium chloride. Such by-products can be largely removed from the aqueous suspension for example by means of a membrane process such as nano-, ultra-, micro- or crossflow filtration.

A further preferred embodiment of the method according to the invention is one in which at least one of the process steps a) to c) is carried out continuously.

The present invention further provides aqueous suspensions of surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, and the surface modification comprises a coating with at least one nonionic dispersant, obtainable by the method described above.

According to a preferred embodiment of the invention, the surface-modified nanoparticulate particles in the aqueous suspensions are coated with a nonionic dispersant, which is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

The present invention further provides the use of aqueous suspensions of surface-modified nanoparticulate particles at least of one metal oxide, metal hydroxide and/or metal oxide hydroxide, in particular titanium dioxide or zinc oxide, which are produced by the method according to the invention as UV protectants in cosmetic sunscreen preparations, as stabilizer in plastics and as antimicrobial active ingredient.

By reference to the examples below, the aim is to illustrate the invention in more detail.

### EXAMPLE 1

### Preparation of Nanoparticulate Zinc Oxide in the Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 27.26 g of zinc chloride per liter and had a zinc ion concentration of 0.2 mol/l. Moreover, solution 1 also comprised 4 g/l of Cremophor® CO 40.

Solution 2 comprised 16 g of sodium hydroxide per liter and thus had a hydroxyl ion concentration of 0.4 mol/l.

4 l of solution 1 were initially introduced into a glass reactor with a total volume of 12 l and stirred (250 rpm). Using a HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and heated to a temperature of 85° C. in a downstream heat exchanger over the course of 1 minute. The suspension obtained then flowed through a second heat exchanger where the suspension was kept at 85° C. for a further 30 seconds. The suspension then flowed successively through a third and fourth heat exchanger where the suspension was cooled to room temperature over the course of a further minute.

The freshly prepared suspension was thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sigma 3K30, 20 000 rpm, 40 700 g) with subsequent drying at 50° C.

The powder obtained had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size which is between 16 nm [for the (102) reflection] and 57 nm [for the (002) reflection]. In transmission electron microscopy (TEM), the resulting powder had an average particle size of 50 to 100 nm.

### EXAMPLE 2

### Continuous Preparation of Nanoparticulate Zinc Oxide in the Presence of Cremophor® CO 40 (Addition Product of 40 Equivalents of Ethylene Oxide onto Hydrogenated Castor Oil)

5 l of water at a temperature of 25° C. were added to a glass reactor with a total volume of 8 l and this was stirred at a rotational speed of 250 rpm. With further stirring, solutions 1 and 2 from Example 1 were continuously metered into the initial charge of water using two HPLC pumps (Knauer, model K 1800, pump head 500 ml/min) via two separate feed tubes, in each case with a metering rate of 0.48 l/min. During this, a white suspension formed in the glass reactor. At the same time, a suspension stream of 0.96 l/min was pumped out of the glass reactor via a riser tube by means of a gear pump (Gather Industrie GmbH, D-40822 Mettmann) and heated to a temperature of 85° C. in a downstream heat exchanger over the course of 1 minute. The resulting suspension then flowed through a second heat exchanger, where the suspension was kept at 85° C. for a further 30 seconds. The suspension then flowed successively through a third and fourth heat exchanger, where the suspension was cooled to room temperature over the course of a further minute.

The freshly produced suspension was thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sigma 3K30, 20 000 rpm, 40 700 g) with subsequent drying at 50° C.

The resulting powder had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size, which is between 16 nm [for the (102) reflection] and 57 nm [for the (002) reflection]. In transmission electron microscopy (TEM), the resulting powder had an average particle size of from 50 to 100 nm.

### EXAMPLE 3

### Preparation of Nanoparticulate Zinc Oxide in the Presence of Cremophor® A 25 (Addition Product of 25 Equivalents of Ethylene Oxide onto Cetylstearyl Alcohol)

Firstly, two aqueous solutions 1 and 2 were prepared. Solution 1 comprised 27.26 g of zinc chloride per liter and had a zinc ion concentration of 0.2 mol/l. Moreover, solution 1 also comprised 4 g/l of Cremophor® A 25.

Solution 2 comprised 16 g of sodium hydroxide per liter and thus had a hydroxyl ion concentration of 0.4 mol/l.

4 l of solution 1 were initially introduced into a glass reactor with a total volume of 12 l and stirred (250 rpm). Using a HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and heated to a temperature of 85° C. in a downstream heat exchanger over the course of 1 minute. The resulting suspension then flowed through a second heat exchanger, where the suspension was kept at 85° C. for a further 30 seconds. The suspension then flowed successively through a third and fourth heat exchanger, where the suspension was cooled to room temperature over the course of a further minute.

The freshly prepared suspension was thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sigma 3K30, 20 000 rpm, 40 700 g) with subsequent drying at 50° C.

The resulting powder had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size, which is between about 15 nm [for the (102) reflection] and about 60 nm [for the (002) reflection]. In transmission electron microscopy (TEM), the resulting powder had an average particle size of from 50 to 100 nm.

### EXAMPLE 4

### Continuous Preparation of Nanoparticulate Zinc Oxide in the Presence of Cremophor® A 25 (Addition Product of 25 Equivalents of Ethylene Oxide onto Cetylstearyl Alcohol)

5 l of water at a temperature of 25° C. were added to a glass reactor with a total volume of 8 l and stirred at a rotational speed of 250 rpm. With further stirring, solutions 1 and 2 from Example 1 were continuously metered into the initial charge of water using two HPLC pumps (Knauer, model K 1800, pump head 500 ml/min) via two separate feed tubes, in each case with a metering rate of 0.48 l/min. During this, a white suspension formed in the glass reactor. At the same time, a suspension stream of 0.96 l/min was pumped out of the glass reactor via a riser tube by means of a gear pump (Gather Industrie GmbH, D-40822 Mettmann) and heated to a temperature of 85° C. in a downstream heat exchanger over the course of 1 minute. The resulting suspension then flowed through a second heat exchanger, where the suspension was kept at 85° C. for a further 30 seconds. The suspension then flowed successively through a third and fourth heat exchanger, where the suspension was cooled to room temperature over the course of a further minute.

The freshly prepared suspension was thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sigma 3K30, 20 000 rpm, 40 700 g) with subsequent drying at 50° C.

The resulting powder had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size, which is between about 15 nm [for the (102) reflection] and about 60 nm [for the (002) reflection]. In transmission electron microscopy (TEM), the resulting powder had an average particle size of from 50 to 100 nm.

### EXAMPLE 5

### Discontinuous Preparation of Nanoparticulate Zinc Oxide in the Presence of Cremophor® A 25 (Addition Product of 25 Equivalents of Ethylene Oxide onto Cetylstearyl Alcohol)

1000 ml of a 0.4M zinc nitrate solution were heated to 40° C. with stirring. Over the course of 6 minutes, 1000 ml of a 0.8M sodium hydroxide solution, likewise heated to 40° C. and which additionally comprised 4 g/l of Cremophor® A 25, were metered in and stirred for a further 2 hours. The precipitated surface-modified product was filtered off, washed with water and the filter cake was dried at 80° C. in a drying cabinet. The powder obtained had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size, which is between 15 nm [for the (102) reflection] and 42 nm [for the (002) reflection]. In transmission electron microscopy (TEM), the resulting powder had an average particle size of from 50 to 100 nm.

### EXAMPLE 6

### Discontinuous Preparation of Nanoparticulate Zinc Oxide in the Presence of Cremophor® A 25 (Addition Product of 25 Equivalents of Ethylene Oxide onto Cetylstearyl Alcohol)

1000 ml of a 0.4M zinc nitrate solution which additionally also comprised 2 g/l of Cremophor A 25 were heated to 40° C. with stirring. Over the course of 6 minutes, 1000 ml of a 0.8M sodium hydroxide solution, likewise heated to 40° C. and which additionally comprised 2 g/l of Cremophor® A 25, were metered in and stirred for a further 2 hours. The precipitated surface-modified product was filtered off, washed with water and the filter cake was dried at 80° C. in a drying cabinet. The powder obtained had, in the UV-VIS spectrum, the absorption band characteristic of zinc oxide at about 350-360 nm. In agreement with this, the X-ray diffraction of the powder showed exclusively the diffraction reflections of hexagonal zinc oxide. The half-width of the X-ray reflections was used to calculate a crystallite size, which is between 17 nm [for the (102) reflection] and 45 nm [for the (002) reflection]. In transmission electron microscopy (TEM), the resulting powder had an average particle size of from 40 to 80 nm.

### EXAMPLE 7

### Use of Nanoparticulate Zinc Oxide Prepared According to Example 1 for Producing a Sunscreen Lotion Comprising 5% by Weight of Zinc Oxide

| | % | Constituents | INCI |
|---|---|---|---|
| A | 7.50 | Uvinul MC 80 | Ethylhexyl Methoxycinnamate |
| | 1.50 | Tween 20 | Polysorbate-20 |
| | 3.00 | Pationic 138 C | Sodium Lauroyl Lactylate |
| | 1.00 | Cremophor CO 40 | PEG-40 Hydrogenated Castor Oil |
| | 1.00 | Cetiol SB 45 | Butyrospermum Parkii (Shea Butter) |
| | 6.50 | Finsolv TN | C12-15 Alkyl Benzoate |
| B | 5.00 | Zinc oxide from | Zinc Oxide |
| | | Ex. 1 | |
| C | 4.00 | Glycerol 87% | Glycerin |
| | 1.00 | D-Panthenol 50 P | Panthenol, Propylene Glycol |
| | 0.30 | Keltrol | Xanthan Gum |
| | 0.10 | Edeta BD | Disodium EDTA |
| | 2.00 | Urea | Urea |
| | 2.00 | Simulgel NS | Hydroxyethyl Acrylate/Sodium |
| | | | Acryloyldimethyl Taurate Copolymer, |
| | | | Squalane, Polysorbate 60 |
| | 64.10 | Water dem. | Aqua dem. |
| D | 0.50 | Lactic Acid | Lactic Acid |
| | 0.50 | Euxyl K 300 | Phenoxyethanol, Methylparaben, |
| | | | Butylparaben, Ethylparaben, |
| | | | Propylparaben, Isobutylparaben |

Phase A is heated to 80° C., then phase B is added, the mixture is homogenized for 3 minutes. Separately, phase C is heated to 80° C. and stirred into the mixture of phases A and B. The mixture is cooled to 40° C. with stirring, then phase D is added. The lotion is briefly afterhomogenized.

### EXAMPLE 8

### Preparation of Nanoparticulate Zinc Oxide in The Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 54.52 g of zinc chloride per liter and had a zinc ion concentration of 0.4 mol/l. Moreover, solution 1 also comprised 4 g/l of Cremophor® CO 40.

Solution 2 comprised 32 g of sodium hydroxide per liter and thus had a hydroxyl ion concentration of 0.8 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 9

### Preparation of Nanoparticulate Zinc Oxide in the Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 54.52 g of zinc chloride per liter and had a zinc ion concentration of 0.4 mol/l. Moreover, solution 1 also comprised 8 g/l of Cremophor® CO 40.

Solution 2 comprised 32 g of sodium hydroxide per liter and thus had a hydroxyl ion concentration of 0.8 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 10

### Preparation of Nanoparticulate Zinc Oxide in the Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 27.26 g of zinc chloride per liter and had a zinc ion concentration of 0.2 mol/l. Moreover, solution 1 also comprised 2 g/l of Cremophor® CO 40.

Solution 2 comprised 16 g of sodium hydroxide per liter and thus had a hydroxyl ion concentration of 0.4 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 11

### Preparation of Nanoparticulate Zinc Oxide in the Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 27.26 g of zinc chloride per liter and had a zinc ion concentration of 0.2 mol/l. Moreover, solution 1 also comprised 4 g/l of Cremophor® CO 40.

Solution 2 comprised 16 g of sodium hydroxide per liter and thus had a hydroxyl ion concentration of 0.4 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 12

### Preparation of Nanoparticulate Zinc Oxide in the Presence of Cremophor® CO 40 (Addition Product of 40 Equivalents of Ethylene Oxide onto Hydrogenated Castor Oil)

Firstly, two aqueous solutions 1 and 2 were prepared. Solution 1 comprised 27.26 g of zinc chloride per liter and had a zinc ion concentration of 0.2 mol/l. Moreover, solution 1 also comprised 8 g/l of Cremophor® CO 40.

Solution 2 comprised 16 g of sodium hydroxide per liter and thus had a hydroxyl ion concentration of 0.4 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 13

### Preparation of Nanoparticulate Zinc Oxide in the Presence of Cremophor® A 25 (Addition Product of 25 Equivalents of Ethylene Oxide onto Cetylstearyl Alcohol)

Firstly, two aqueous solutions 1 and 2 were prepared. Solution 1 comprised 54.52 g of zinc chloride per liter and had an zinc ion concentration of 0.4 mol/l. Moreover, solution 1 also comprised 2 g/l of Cremophor® A 25.

Solution 2 comprised 32 g of sodium hydroxide per liter and thus had a hydroxyl ion concentration of 0.8 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 14

### Preparation of Nanoparticulate Zinc Oxide in the Presence of Cremophor® A 25 (Addition Product of 25 Equivalents of Ethylene Oxide onto Cetylstearyl Alcohol)

Firstly, two aqueous solutions 1 and 2 were prepared. Solution 1 comprised 54.52 g of zinc chloride per liter and had a zinc ion concentration of 0.4 mol/l. Moreover, solution 1 also comprised 4 g/l of Cremophor® A 25.

Solution 2 comprised 32 g of sodium hydroxide per liter and thus had a hydroxyl ion concentration of 0.8 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

### EXAMPLE 15

### Preparation of Nanoparticulate Zinc Oxide in the Presence of Cremophor® A 25 (Addition Product of 25 Equivalents of Ethylene Oxide onto Cetylstearyl Alcohol)

Firstly, two aqueous solutions 1 and 2 were prepared. Solution 1 comprised 54.52 g of zinc chloride per liter and had a zinc ion concentration of 0.4 mol/l. Moreover, solution 1 also comprised 8 g/l of Cremophor® A 25.

Solution 2 comprised 32 g of sodium hydroxide per liter and thus had a hydroxyl ion concentration of 0.8 mol/l.

4 l of solution 1 were initially introduced into a glass reactor with a total volume of 12 l and stirred (250 rpm). Using an HPLC pump (Knauer, model K 1800, pump head 1000 ml/min), 4 l of solution 2 were metered into the stirred solution over the course of 6 minutes at room temperature. During this, a white suspension formed in the glass reactor.

Immediately after the metered addition was complete, a suspension stream of 0.96 l/min was pumped off from the resulting suspension via a riser tube by means of a gear pump (Gather Industrie GmbH, D-40822 Mettmann) and transferred to a heat exchanger heated at 85° C. and with a volume of 0.96 l. The heat exchanger is preheated to the desired temperature with hot water before being deployed. The suspension then flowed successively through a second and third heat exchanger where the suspension was cooled to room temperature over the course of a further minute.

The freshly prepared suspension was washed and thickened by a factor of 15 in a crossflow ultrafiltration laboratory installation (Sartorius, model SF Alpha, PES cassette, Cut off 100 kD). Subsequent isolation of the solid powder was carried out using an ultracentrifuge (Sorvall RC 6, Thermo Electron Corporation, 13 000 rpm) with subsequent drying at 80° C.

The powder obtained had, in the UV-VIS spectrum, the absorption band characteristic of zinc oxide at about 350-360 nm. In transmission electron microscopy (TEM), the resulting powder had an average particle size of 50 to 100 nm.

1. A method of producing surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, comprising the steps
a) producing a solution of water and at least one metal salt of the abovementioned metals (solution 1) and a solution of water and at least one strong base (solution 2), where at least one of the two solutions 1 and 2 comprises at least one nonionic dispersant whose chemical structure comprises between 2 and 10 000 —CH₂CH₂O— groups,
b) mixing the solutions 1 and 2 produced in step a) at a temperature in the range from 0 to 120° C., during which the surface-modified nanoparticulate particles are formed and precipitate out of the solution to form an aqueous suspension,
c) separating off the surface-modified nanoparticulate particles from the aqueous suspension obtained in step b), and
d) drying the surface-modified nanoparticulate particles obtained in step c).

2. The method according to claim 1, wherein the metal salt is zinc chloride, zinc nitrate, zinc acetate or titanium tetrachloride.

3. The method according to one of claims 1, wherein the strong base is an alkali metal hydroxide, an alkaline earth metal hydroxide or ammonia.

4. The method according to claim 1, wherein the nonionic dispersant comprises in its chemical structure between 3 and 200 —CH₂CH₂O— groups.

5. The method according to claim 1, wherein the nonionic dispersant comprises one or more connected ethylene glycol chains whose chemical structure corresponds to the formula —(—CH₂CH₂O—)ₙ— where n is from about 2 to about 80.

6. The method according to claim 1, wherein the nonionic dispersant is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

7. The method according to claim 1, wherein at least one of process steps a) to d) is carried out continuously.

8. A surface-modified nanoparticulate particle at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, and the surface modification comprises a coating with at least one nonionic dispersant, obtainable by a method according to claim 1.

9. A surface-modified nanoparticulate particle at least of one metal oxide, metal hydroxide and/or metal oxide hydroxide, where the surface modification comprises a coating with a nonionic dispersant, with a BET surface area in the range from 25 to 500 m²/g.

10. The surface-modified nanoparticulate particle according to claim 8, where the nonionic dispersant is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

11. (canceled)

12. (canceled)

13. A method of producing an aqueous suspension of surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, comprising the steps
a) producing a solution of water and at least one metal salt of the abovementioned metals (solution 1) and a solution of water and at least one strong base (solution 2), where at least one of the two solutions 1 and 2 comprises at least one nonionic dispersant whose chemical structure comprises between 2 and 10 000 —CH₂CH₂O— groups,
b) mixing the solutions 1 and 2 produced in step a) at a temperature in the range from 0 to 120° C., during which the surface-modified nanoparticulate particles are formed and precipitate out of the solution to form an aqueous suspension, and
c) if appropriate concentrating the formed aqueous suspension and/or separating off by-products.

14. The method according to claim 13, wherein the metal salt is zinc chloride, zinc nitrate, zinc acetate or titanium tetrachloride.

15. The method according to claim 13, wherein the strong base is an alkali metal hydroxide, an alkaline earth metal hydroxide or ammonia.

16. The method according to claim 13, wherein the nonionic dispersant comprises between 3 and 200 —CH₂CH₂O— groups in its chemical structure.

17. The method according to claim 13, wherein the nonionic dispersant comprises one or more connected ethylene glycol chains whose chemical structure corresponds to the formula —(—CH₂CH₂O—)ₙ— where n is from about 2 to about 80.

18. The method according to claim 13, wherein the nonionic dispersant is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

19. The method according to claim 13, wherein at least one of the process steps a) to c) is carried out continuously.

20. An aqueous suspension of surface-modified nanoparticulate particles at least of one metal oxide, metal hydroxide and/or metal oxide hydroxide, where the metal or the metals are chosen from the group consisting of aluminum, magnesium, cerium, iron, manganese, cobalt, nickel, copper, titanium, zinc and zirconium, and the surface modification comprises a coating with at least one nonionic dispersant, obtainable by a method according to claim 13.

21. The aqueous suspension according to claim 20, where the nonionic dispersant is an addition product of from 2 to 80 mol of ethylene oxide onto linear fatty alcohols having 8 to 22 carbon atoms, onto alkylphenols having 8 to 15 carbon atoms in the alkyl group or onto castor oil and/or hydrogenated castor oil.

22. (canceled)

23. (canceled)

24. A cosmetic sunscreen preparation comprising the surface-modified nanoparticulate particles according to claim 1 as a UV protectant.

25. A plastics composition comprising the surface-modified nanoparticulate particles according to claim 1 as a stabilizer.

26. An antimicrobial active ingredient comprising the surface-modified nanoparticulate particles according to claim 1.
